(19) United States
(12) Patent Application Publication
(10) Pub. No.: US 2020/0022847 A1
(43) Pub. Date: Jan. 23, 2020

(54) STERILE MOUTH-INSERTABLE DEVICE TO STOP A NOSEBLEED

(71) Applicant: Douglas Guion, Emporia, KS (US)

(72) Inventor: Douglas Guion, Emporia, KS (US)

(21) Appl. No.: 16/041,813

(22) Filed: Jul. 22, 2018

(51) Int. Cl. A61F 13/20; A61F 13/12

(52) U.S. Cl. CPC A61F 13/2008; A61F 13/126; A61F 15/001; A61F 13/2057; A61F 13/2028

(57) ABSTRACT

A mouth-insertable device, including a main body having a cylindrical shape, a first end disposed at an end of the main body, and a second end disposed at another end of the main body opposite from the first end, wherein the main body has a size to fit between an inner upper lip and an upper gum line of a user.

### BACKGROUND

### 1. Field

The present general inventive concept relates generally to a medical device, and particularly, to a sterile medical and mouth-insertable device that effectively stops a nose from bleeding.

### 2. Description of the Related Art

For many individuals running or bleeding noses can create discomfort and require the embarrassment of sticking tissues in the nose in order to absorb the blood or mucus. Currently, on the market, there are products for absorbing blood after nosebleeds, but these options are often too obvious or lack the ability to halt the bloodflow from the nose altogether.

Therefore, there is a need for a device that effectively stops a nose from bleeding.

### SUMMARY

The present general inventive concept provides a sterile mouth-insertable device to stop a nosebleed.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a mouth-insertable device, including a main body having a cylindrical shape, a first end disposed at an end of the main body, and a second end disposed at another end of the main body opposite from the first end, wherein the main body has a size to fit between an inner upper lip and an upper gum line of a user.

The main body may be constructed from deformably elastic material.

The first end and the second end may each have at least one of a flat shape, a convex shape, and a concave shape.

Inserting the mouth-insertable device between the inner upper lip and the upper gum line of the user may stop a nose of the user from bleeding.

The mouth-insertable device may be sterile.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a sterile mouth-insertable device to stop a nosebleed, along with a wrapper and a packaging, according to an exemplary embodiment of the present general inventive concept; and

FIG. 2 illustrates the sterile mouth-insertable device to stop a nosebleed as utilized by a user, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

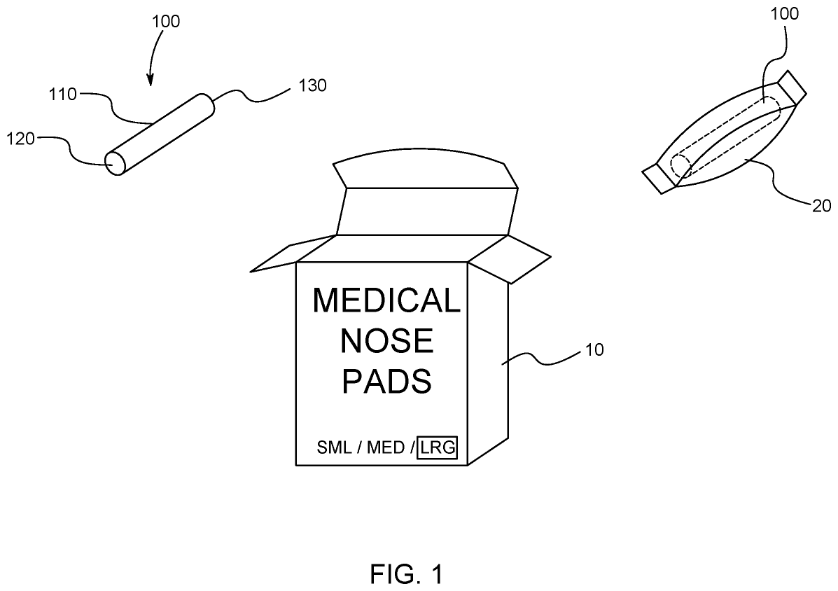

FIG. 1 illustrates a sterile mouth-insertable device 100 to stop a nosebleed, along with a wrapper 20 and a packaging 10, according to an exemplary embodiment of the present general inventive concept.

The sterile mouth-insertable device 100 may have a cylindrical (i.e., tubular) shape, or any type of shape known to one of ordinary skill in the art to be able to fit inside a user's mouth, and may be constructed from Styrofoam, padding, sponge material, plastic, rubber, metal, wood, cloth, etc., but is not limited thereto. As such, the main body 110 may constructed from deformably elastic material, such that the main body 110 may be temporarily deformed, and then may return to its original shape.

Also, the sterile mouth-insertable device 100 may have various sizes depending on a size of the user's mouth.

A packaging 10 may house a plurality of sterile mouth-insertable devices 100, and each sterile mouth-insertable device 100 may be individually wrapped in a wrapper 20 to maintain sterility.

As illustrated in FIG. 1, the table 100 may include a main body 110, a first end 120, and a second 130, but is not limited thereto.

The main body 110 may have a cylindrical shape, and thus, may be rollable when placed on a surface.

The main body 110 may also be constructed from material that is slightly elastic and/or deformable, so that the sterile mouth-insertable device 100 may be comfortably inserted in the user's mouth.

The main body 110 may include the first end 120 and the second end 130 disposed on opposite ends of the main body 110.

The first end 120 and the second end 130 may be flat, concave, or convex, or any combination thereof.

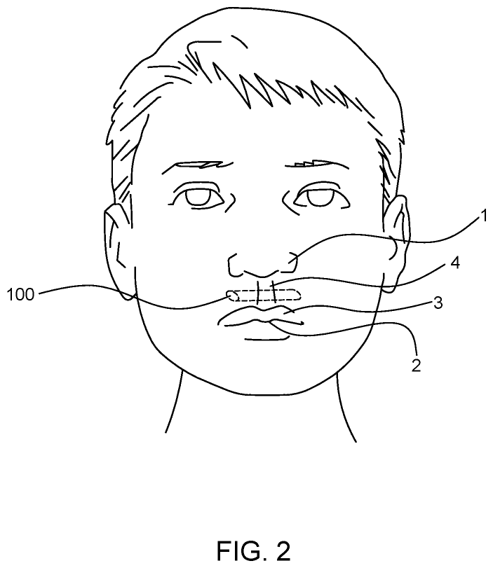

FIG. 2 illustrates the sterile mouth-insertable device to stop a nosebleed as utilized by a user, according to an exemplary embodiment of the present general inventive concept.

When a nose 1 of a user is bleeding, the 100 may be placed inside a mouth 2 of the user, specifically, at an inner portion of the mouth 2 of above an upper lip 3 of the user, until the 100 makes contact with a maxillary labial frenum (i.e., the connector between an upper gumline and an inner portion of the upper lip) of the user. In other words, the 100 may be disposed inside the mouth 2 of the user, at a gum line of the user, and directly behind a philtrum 4 of the user.

The 100 may cause an outer area above the upper lip 2 of the user (including the philtrum 4) to protrude slightly, thereby applying an inherent pressure to a lower portion of the nose 1. As a result of the proper placement of the 100 inside the mouth 2 of the user, the bleeding from the nose 1 may cease within a few seconds.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A mouth-insertable device, comprising:
a main body having a cylindrical shape;
a first end disposed at an end of the main body; and
a second end disposed at another end of the main body opposite from the first end, wherein the main body has a size to fit between an inner upper lip and an upper gum line of a user.

2. The mouth-insertable device of claim 1, wherein the main body is constructed from deformably elastic material.

3. The mouth-insertable device of claim 1, wherein the first end and the second end each have at least one of a flat shape, a convex shape, and a concave shape.

4. The mouth-insertable device of claim 1, wherein inserting the mouth-insertable device between the inner upper lip and the upper gum line of the user stops a nose of the user from bleeding.

5. The mouth-insertable device of claim 1, wherein the mouth-insertable device is sterile.
